(19) United States
(12) Patent Application Publication
(10) Pub. No.: US 2025/0090233 A1
(43) Pub. Date: Mar. 20, 2025

(54) METHOD AND APPARATUS FOR DETERMINING HEART VALVE BASED ON MULTIPLE PLANES, AND ELECTRONIC DEVICE

(71) Applicant: VENUS MEDTECH (HANGZHOU) INC., Hangzhou (CN)

(72) Inventors: Wenjun Huang, Hangzhou (CN); Wei Chen, Hangzhou (CN); Xu Zhao, Hangzhou (CN); Xiaotong Zeng, Hangzhou (CN); Xiaodong Zhan, Hangzhou (CN); Wenwen Wu, Hangzhou (CN)

(21) Appl. No.: 18/964,725

(22) Filed: Dec. 2, 2024

Related U.S. Application Data
(63) Continuation of application No. PCT/CN2023/090356, filed on Apr. 24, 2023.

(30) Foreign Application Priority Data
Jun. 2, 2022 (CN) 202210629414.5

(51) Int. Cl. A61B 34/10; A61F 2/24; G06T 7/00; G06T 7/62; G06T 7/73

(52) U.S. Cl. CPC A61B 34/10; A61F 2/24; G06T 7/0012; G06T 7/62; G06T 7/73; A61B 2034/108; G06T 2207/30048

(57) ABSTRACT

Disclosed are a method, an apparatus, and an electronic device for determining a heart valve based on multiple planes. The method includes steps of: determining a plurality of target planes in a heart based on an anatomical image; determining at least one first initial pushing point located on an inner contour line of a heart valve leaflet in each target plane; determining at least one first pushing reach point corresponding to the at least one first initial pushing point; determining at least one first pushing range of each target plane based on the at least one first pushing reach point; determining a second pushing range of the heart valve based on the at least one first pushing range; and determining an artificial heart valve that matches the heart valve based on the second pushing range.

### CROSS-REFERENCE TO RELATED APPLICATIONS

The present application is a Continuation Application of PCT Application No. PCT/CN2023/090356, filed on Apr. 24, 2023, which claims the priority of Chinese Patent Application No. 202210629414.5, filed on Jun. 2, 2022, the entire contents of which are hereby incorporated by reference.

### TECHNICAL FIELD

The present disclosure relates to the field of medical technology, and in particular to a method, an apparatus, and an electronic device for determining a heart valve based on multiple planes.

### BACKGROUND

The aortic valve which is located within the opening between the left ventricle and the ascending aorta, will open with the contraction of the left ventricle. A normal aortic valve consists of three semilunar leaflets, allowing blood to flow through the aortic valve into the ascending aorta to the entire body. When there is congenital malformation or degenerative calcification disease with the aortic valve leaflets, the leaflets may thicken, calcify, or fuse, resulting in a narrowed valve opening. This reduces the blood flow pumped into the aorta, impairing normal cardiac function and, in severe cases, leading to heart failure.

For patients with severe aortic stenosis, surgical replacement of the diseased valve is the optimal treatment. Traditional surgical replacement methods involve significant surgical trauma and lengthy recovery periods. Currently, an increasing number of patients undergo transcatheter aortic valve replacement (TAVR). During TAVR, it is essential to select an artificial heart valve that matches the patient's specific physiological and anatomical structure, i.e., the appropriate model of the artificial heart valve.

When determining the model of the artificial heart valve, the open extent to which the patient's heart valve can be pushed is a critical reference parameter. Currently, there is no standardized procedure for determining the opening extent of heart valve during TAVR. In most cases, it depends on the physician's experience, leading to longer determination times and compromised accuracy.

### SUMMARY

The embodiments of the present disclosure provide a method, an apparatus, and an electronic device for determining a heart valve based on multiple planes, aiming to at least solve the technical problem of compromised accuracy, which arises from the experience-based methods for determining heart valves in related art.

According to an aspect of the embodiments of the present disclosure, the present disclosure provides a method for determining a heart valve based on multiple planes which includes steps of: determining a plurality of target planes in a heart based on an anatomical image of the heart; determining at least one first initial pushing point in each target plane of the plurality of target planes, wherein each first initial pushing point is a point located on an inner contour line of a heart valve leaflet; determining at least one first pushing reach point corresponding to the at least one first initial pushing point, wherein each first pushing reach point corresponds to a position where a corresponding first initial pushing point reaches after moving a first pushing distance towards a vessel wall, and the vessel wall is a wall of an aortic sinus in the heart; determining at least one first pushing range of each target plane based on the at least one first pushing reach point; determining a second pushing range of the heart valve based on the at least one first pushing range of each target plane; and determining an artificial heart valve that matches the heart valve based on the second pushing range.

In some embodiments, the step of determining a plurality of the target planes in a heart based on the anatomical image of the heart includes steps of: determining a position of a reference plane in the heart based on the anatomical image; and selecting a plurality of cross-sectional planes above the reference plane, wherein the plurality of cross-sectional planes is defined as the plurality of target planes.

In some embodiments, the reference plane is a plane determined by a lowest point where the heart valve leaflet attaches to the wall of the aortic sinus.

In some embodiments, the step of determining at least one first pushing range of each target plane based on the at least one first pushing reach point includes steps of: determining a target pattern based on the at least one first pushing reach points, wherein each first pushing reach point is located on a contour line of the target pattern; and determining size information of the target pattern and determining the at least one first pushing range based on the size information.

In some embodiments, the size information of the target pattern comprises at least one of a perimeter, an area, a radius, a diameter, and an average diameter of the target pattern.

In some embodiments, the step of determining a target pattern based on the at least one first pushing reach points includes steps of: determining a structural feature of the heart valve in each target plane, wherein the structural feature of the heart valve includes a fusion situation between any two adjacent leaflets of the heart valve; determining at least one second pushing reach point in each target plane based on the structural feature of the heart valve, wherein each second pushing reach point is located at a boundary edge between the two adjacent leaflets; and determining the target pattern based on the at least one first pushing reach point and the at least one second pushing reach point, wherein each second pushing reach point is located on the contour line of the target pattern.

In some embodiments, the step of determining at least one second pushing reach point in each target plane based on the structural feature of the heart valve includes steps of: determining a junction point of the two adjacent leaflets as a second pushing reach point corresponding to the two adjacent leaflets in case of no fusion between the two adjacent leaflets; determining a second initial pushing point at a fusion area of the two adjacent leaflets in case of fusion occurring between the two adjacent leaflets; and determining a second pushing reach point based on the second initial pushing point.

In some embodiments, the step of determining a second pushing reach point based on the second initial pushing point includes steps of: determining the fusion situation between the two adjacent leaflets, wherein the fusion situation includes at least one of a fusion type and a length of a fused portion; determining a second pushing distance corresponding to the second initial pushing point based on the fusion situation; and determining the second pushing reach point based on the second pushing distance and the second initial pushing point, wherein the second pushing reach point corresponds to a position where the second initial pushing point reaches after moving the second pushing distance along the fused portion in a direction away from a center of the heart valve.

In some embodiments, the step of determining the fusion situation between the two adjacent leaflets includes: determining the fusion type and the length of the fused portion between two adjacent leaflets; and wherein the step of determining a second pushing distance corresponding to the second initial pushing point based on the fusion situation includes steps of: determining a pushing degree at the fusion area based on the fusion type; and determining the second pushing distance based on the pushing degree and the length of the fused portion.

In some embodiments, the fusion situation includes at least one of a fusion type and a length of a fused portion, and the fusion type includes fully calcified fusion, partially calcified fusion, and non-calcified fusion.

In some embodiments, the step of determining a second pushing range of the heart valve based on the at least one first pushing range of each target plane includes: determining a smallest first pushing range of the at least one first pushing ranges as the second pushing range.

In some embodiments, the step of determining at least one first pushing reach point corresponding to the at least one first initial pushing point includes steps of: determining a thickness and a calcification degree of the leaflet corresponding to each first initial pushing point; determining each first pushing distance based on the thickness and the calcification degree of the leaflet; and determining a position of each first pushing reach point based on a position of each first initial pushing point and each first pushing distance.

In some embodiments, the step of determining each first pushing distance based on the thickness and the calcification degree of the leaflet includes steps of: determining a distance between an inner side of the leaflet where each first initial pushing point is located and the vessel wall; and determining a distance coefficient based on the thickness and calcification degree of the leaflet, and calculating each first pushing distance by multiplying the distance between the inner side of the leaflet and the vessel wall by the distance coefficient.

In some embodiments, each target plane is perpendicular to a central axis of an aorta of the heart.

In some embodiments, the anatomical image includes structures of an aortic valve and surrounding tissues, and each target plane comprises contour information of the heart valve and the surrounding tissues.

According to an aspect of the embodiments of the present disclosure, the present disclosure provides a method for determining a heart valve based on multiple planes which includes steps of: acquiring an anatomical image of a heart; determining a plurality of target planes in the heart based on the anatomical image of the heart; determining at least one first initial pushing point in each target plane of the plurality of target planes, wherein each first initial pushing point is a point located on an inner contour line of a heart valve leaflet; determining at least one first pushing reach point corresponding to the at least one first initial pushing point, wherein each first pushing reach point corresponds to a position where a corresponding first initial pushing point reaches after moving a first pushing distance towards a vessel wall, and the vessel wall is a wall of an aortic sinus in the heart; determining at least one first pushing range of each target plane based on the first pushing reach point; determining a second pushing range of the heart valve based on the at least one first pushing range of each target plane; determining a target setting parameter of an artificial heart valve based on the second pushing range, wherein the target setting parameter includes dimensional information of the artificial heart valve; and determining the artificial heart valve based on the target setting parameter.

In some embodiments, the target plane is a plane located above an aortic valve annulus, and the step of determining a target setting parameter of an artificial heart valve based on the second pushing range includes steps of determining a first structural feature of the aortic valve annulus and a second structural feature of a heart valve below the aortic valve annulus; and determining the target setting parameter based on the first structural feature, the second structural feature and the second pushing range.

According to an aspect of the embodiments of the present disclosure, the present disclosure provides an apparatus for determining a heart valve based on multiple planes which includes a processing module, configured to determine a plurality of target planes in a heart based on an anatomical image of the heart; an identification module, configured to determine at least one first initial pushing point in each target plane of the plurality of target planes, wherein each first initial pushing point is a point located on an inner contour line of a heart valve leaflet; a positioning module, configured to determine at least one first pushing reach point corresponding to the at least one first initial pushing point, wherein each first pushing reach point corresponds to a position where a corresponding first initial pushing point reaches after moving a first pushing distance towards a vessel wall, and the vessel wall is a wall of an aortic sinus in the heart; a calculation module, configured to determine at least one first pushing range of each target plane based on the at least one first pushing reach point; a selection module, configured to determine a second pushing range of the heart valve based on the at least one first pushing range of each target plane; and a determination module, configured to determine an artificial heart valve that matches the heart valve based on the second pushing range.

According to an aspect of the embodiments of the present disclosure, the present disclosure provides a non-volatile storage medium including a stored program. When the program is executed, a device including the non-volatile storage medium is controlled to perform the above-mentioned method for determining the heart valve based on multiple planes.

According to an aspect of the embodiments of the present disclosure, the present disclosure provides an electronic device including a processor. The processor is configured to execute a program, and when the program is executed, the above-mentioned method for determining the heart valve based on multiple planes is implemented.

In the embodiments of the present disclosure, a plurality of target planes in a heart are determined based on an anatomical image of the heart; at least one first initial pushing point in each target plane of the plurality of target planes is determined, wherein each first initial pushing point is a point located on an inner contour line of a heart valve leaflet; at least one first pushing reach point corresponding to the at least one first initial pushing point is determined, wherein each first pushing reach point corresponds to a position where a corresponding first initial pushing point reaches after moving a first pushing distance toward a vessel wall, and the vessel wall is a wall of an aortic sinus in the heart; at least one first pushing range of each target plane is determined based on the at least one first pushing reach point; a second pushing range of the heart valve is determined based on the at least one first pushing range of each target plane; and an artificial heart valve that matches the heart valve is determined based on the second pushing range. By determining multiple target planes in the heart valve and identifying the initial pushing point and the pushing reach point within each target plane, the first pushing range of each target plane is determined, and further the second pushing range of the heart valve is determined based on the first pushing range of each target plane. In this way, a prosthetic heart valve matching the heart valve is determined based on the second pushing range, thereby solving the technical problem of compromised accuracy which arises from the experience-based methods for determining heart valves in the related art.

### BRIEF DESCRIPTION OF THE DRAWINGS

The accompanying drawings described herein are provided to facilitate the understanding of the present disclosure and constitute a part of this disclosure. The illustrative embodiments and their descriptions are intended to explain the present disclosure and should not be construed as undue limitation thereto. In the drawings:

FIG. 1 is a schematic flowchart of a method for determining a heart valve based on multiple planes according to one embodiment of the present disclosure.

FIG. 2 is a longitudinal section of the heart valve according to one embodiment of the present disclosure.

FIG. 3 is a schematic flowchart of a process for determining the heart valve according to one embodiment of the present disclosure.

FIG. 4 is a schematic flowchart of a process for determining the heart valve according to another embodiment of the present disclosure.

FIG. 5 is a schematic flowchart of a method for determining the heart valve based on multiple planes according to another embodiment of the present disclosure.

FIG. 6a is a cross section of a first type of a heart valve according to one embodiment of the present disclosure.

FIG. 6b is a schematic view illustrating an initial pushing point and a pushing reach point of the first type of the heart valve according to one embodiment of the present disclosure.

FIG. 6c is a schematic view illustrating a pushing range of the first type of the heart valve according to one embodiment of the present disclosure.

FIG. 7a is a cross section of a second type of a heart valve according to one embodiment of the present disclosure.

FIG. 7b is a schematic view illustrating an initial pushing point and a pushing reach point of the second type of the heart valve according to one embodiment of the present disclosure.

FIG. 7c is a schematic view illustrating a pushing range of the second type of the heart valve according to one embodiment of the present disclosure.

FIG. 8a is a cross section of a third type of a heart valve according to one embodiment of the present disclosure.

FIG. 8b is a schematic view illustrating an initial pushing point and a pushing reach point of the third type of the heart valve according to one embodiment of the present disclosure.

FIG. 8c is a schematic view illustrating a pushing range of the third type of the heart valve according to one embodiment of the present disclosure.

FIG. 9a is a cross section of a fourth type of a heart valve according to one embodiment of the present disclosure.

FIG. 9b is a schematic view illustrating an initial pushing point and a pushing reach point of the fourth type of the heart valve according to one embodiment of the present disclosure.

FIG. 9c is a schematic view illustrating a pushing range of the fourth type of the heart valve according to one embodiment of the present disclosure.

FIG. 10a is a cross section of a fifth type of a heart valve according to one embodiment of the present disclosure.

FIG. 10b is a schematic view illustrating an initial pushing point and a pushing reach point of a first subtype of the fifth type of a heart valve according to one embodiment of the present disclosure.

FIG. 10c is a schematic view illustrating a pushing range of the first subtype of the fifth type of the heart valve according to one embodiment of the present disclosure.

FIG. 10d is a schematic view illustrating an initial pushing point and a pushing reach point of a second subtype of the fifth type of a heart valve according to one embodiment of the present disclosure.

FIG. 10e is a schematic view illustrating a pushing range of the second subtype of the fifth type of the heart valve according to one embodiment of the present disclosure.

FIG. 10f is a schematic view illustrating an initial pushing point and a pushing reach point of a third subtype of the fifth type of a heart valve according to one embodiment of the present disclosure.

FIG. 10g is a schematic view illustrating a pushing range of the third subtype of the fifth type of the heart valve according to one embodiment of the present disclosure.

FIG. 10h is a schematic view illustrating an initial pushing point and a pushing reach point of a fourth subtype of the fifth type of a heart valve according to one embodiment of the present disclosure.

FIG. 10i is a schematic view illustrating a pushing range of the fourth subtype of the fifth type of the heart valve according to one embodiment of the present disclosure.

FIG. 11 is a schematic structural view of an apparatus for determining a heart valve based on multiple planes according to one embodiment of the present disclosure.

FIG. 12 is a schematic structural view of a computer device according to one embodiment of the present disclosure.

### DESCRIPTION OF THE EMBODIMENTS

In order to help those skilled in the art better understand the present disclosure, the following will describe the technical solutions of the embodiments of the present disclosure in a clear and complete manner with reference to the accompanying drawings of the embodiments. It is apparently that the described embodiments are only part of the embodiments of the present disclosure, rather than all of the embodiments. Based on the embodiments of the present disclosure, all other embodiments obtained by those skilled in the art without any inventive effort should fall within the scope of protection of the present disclosure.

It should be noted that the terms “first”, “second”, etc., in the specification, claims, and the above-mentioned drawings of the present disclosure are used to distinguish similar objects and are not necessarily used to describe a specific order or sequence. It should be understood that such data can be interchangeable in appropriate circumstances, so that the embodiments of the present disclosure described here can be implemented in a sequence other than the one illustrated or described here. Additionally, the terms “including” and “having” and their variations are intended to cover non-exclusive inclusion, for example, a process, method, system, product, or apparatus that includes a series of steps or units need not be limited to the steps or units explicitly listed but may also include other steps or units inherent to the process, method, product, or apparatus, which are not explicitly listed.

First, the following terms or terminology that are raised in describing the embodiments of the present disclosure are applicable to the interpretations below:

Normal Aortic Structure: The aortic valve is located at the opening between the left ventricle and the ascending aorta. It opens with the contraction of the left ventricle to pump blood into the aorta, which then supplies blood to the entire body. The normal aortic valve consists of three semilunar leaflets. The leaflets are attached to the proximal end of the aortic valve, and the aortic wall bulges outwardly to form the aortic sinus, which includes the left coronary sinus, right coronary sinus, and non-coronary sinus according to the opening of the respective coronary arteries. Each leaflet is attached to the aortic wall with a semicircular leaflet margin, and two adjacent leaflets have a junction at their boundary edges.

Aortic Stenosis: Aortic stenosis (AS) refers to the narrowing of the opening of the aortic valve in the heart, which prevents it from fully opening. Aortic stenosis leads to thickening and narrowing of the valve between the heart's main pumping chamber (the left ventricle) and the body's main artery (the aorta). This narrowing reduces or obstructs the flow of blood from the heart to other parts of the body. Aortic stenosis is typically caused by congenital valve malformations (e.g., bicuspid, quadricuspid, or unicuspid valves) or degenerative valve calcification, often presenting as leaflet calcification, thickening of the leaflets, or fusion at the leaflet junctions.

Leaflet Fusion: Fusion occurs at the junction edges of two adjacent leaflets, accompanied by calcification or non-calcification. The boundary edges of the two leaflets may fuse entirely or partially, preventing full opening and thereby reducing the leaflet opening. Based on the fusion situation, it can be classified into three types: complete calcified fusion, partial calcified fusion, and non-calcified fusion. Non-calcified fusion refers to the adhesion of two adjacent leaflets without accompanying calcification.

Leaflet Inner Side: During ventricular contraction, when the leaflets are fully open, this refers to the side of the leaflet closest to the center axis of the aorta.

Leaflet Outer Side: During ventricular contraction, when the leaflets are fully open, this refers to the side of the leaflet furthest from the center axis of the aorta.

Leaflet Thickness: The distance from the outer side of the leaflet to the inner side of the leaflet at the same cross-sectional height of the leaflet.

Reference Plane: The plane corresponding to the aortic annulus, specifically referring to the plane determined at the lowest point where the aortic valve leaflets attach to the vessel wall. This is typically referred to as the virtual valve annulus plane.

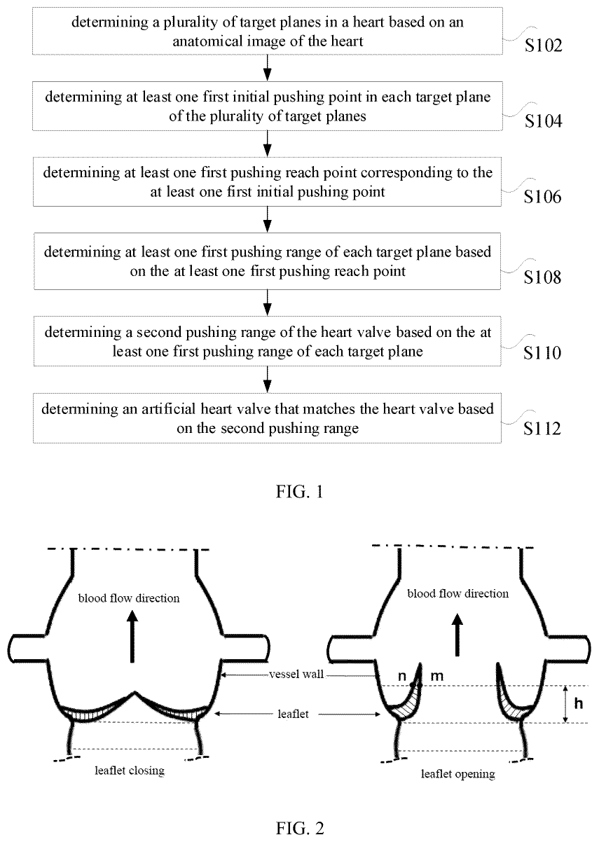

FIG. 1 is a schematic flowchart of a method for determining a heart valve based on multiple planes according to one embodiment of the present disclosure. As shown in FIG. 1, the method includes steps S102 to S112.

S102, determining a plurality of target planes in a heart based on an anatomical image of the heart.

In the technical solution provided in step S102, the multiple target planes are cross-sectional planes of the heart, and each target plane among them is perpendicular to the central axis of the aorta.

Additionally, the anatomical image of the heart includes at least the anatomical images of the aortic valve and its surrounding tissues. Each of the above-mentioned multiple target planes contains at least the contour information of the heart valve and the contour information of the surrounding tissues of the heart valve.

S104, determining at least one first initial pushing point in each target plane of the plurality of target planes, wherein each first initial pushing point is a point located on an inner contour line of a heart valve leaflet;

S106, determining at least one first pushing reach point corresponding to the at least one first initial pushing point, wherein each first pushing reach point corresponds to a position where a corresponding first initial pushing point reaches after moving a first pushing distance towards a vessel wall, and the vessel wall is a wall of an aortic sinus in the heart; and

S108, determining at least one first pushing range of each target plane based on the at least one first pushing reach point.

In the technical solution provided in step S108, a target pattern can be determined in each target plane based on the first pushing reach point, and all the first pushing reach points are located on the contour line of the target pattern. The first pushing range corresponding to each target plane can then be determined based on the size information of the target pattern. The size information of the target pattern includes parameters such as perimeter, area, radius, diameter, and average diameter.

Specifically, when the type of the artificial heart valve is a balloon-expandable valve, the size information of the target pattern may be the area. When the type of the artificial heart valve is a self-expanding valve, the size information of the target pattern may be the perimeter.

S110, determining a second pushing range of the heart valve based on the at least one first pushing range of each target plane.

In the technical solution provided in step S110, a smallest first pushing range among a plurality of first pushing ranges is determined as the second pushing range.

S112, determining an artificial heart valve that matches the heart valve based on the second pushing range.

A plurality of target planes in a heart are determined based on an anatomical image of the heart; at least one first initial pushing point in each target plane of the plurality of target planes is determined, wherein each first initial pushing point is a point located on an inner contour line of a heart valve leaflet; at least one first pushing reach point corresponding to the at least one first initial pushing point is determined, wherein each first pushing reach point corresponds to a position where a corresponding first initial pushing point reaches after moving a first pushing distance toward a vessel wall, and the vessel wall is a wall of an aortic sinus in the heart; at least one first pushing range of each target plane is determined based on the at least one first pushing reach point; a second pushing range of the heart valve is determined based on the at least one first pushing range of each target plane; and an artificial heart valve that matches the heart valve is determined based on the second pushing range. By determining multiple target planes in the heart valve and identifying the initial pushing point and the pushing reach point within each target plane, the first pushing range of each target plane is determined, and further the second pushing range of the heart valve is determined based on the first pushing range of each target plane. In this way, a prosthetic heart valve matching the heart valve is determined based on the second pushing range, thereby solving the technical problem of compromised accuracy which arises from the experience-based methods for determining heart valves in the related art.

In the technical solution provided in step S102, a reference plane is determined based on the anatomical image, and afterward multiple cross sectional planes with equal or unequal intervals above the reference plane are selected as target planes. In addition, each cross-sectional plane among the multiple cross-sectional planes is perpendicular to the central axis of the aorta of the heart. Specifically, during determining a position of a reference plane in the heart based on the anatomical image, a plane is determined by a lowest point where the heart valve leaflet attaches to the wall of the aortic sinus in the anatomical image, and such a plane serves as the reference plane.

Specifically, FIG. 2 is a longitudinal section view of the heart. “h” representing a distance between two horizontal dashed lines in FIG. 2 corresponds to the height of the target plane relative to the reference plane. Point “m” represents the inner side of the leaflet, while point “n” represents the outer side of the leaflet, and the distance between point “m” and point “n” represents the thickness of the leaflet.

The height can be determined based on the actual conditions of the native leaflet, with values taken at equal or unequal intervals above the reference plane. For example, heights of 2 mm, 4 mm, 6 mm, and 8 mm above the reference plane can be selected.

In the technical solution provided in step S108, during determining the first pushing range of each target plane based on the first pushing reach point, a target pattern is determined based on the first pushing reach point, wherein the first pushing reach point is entirely located on a contour line of the target pattern.

As an optional embodiment, for different types of heart valves, during determining the target pattern based on the first pushing reach point, first, a structural feature of the heart valve is determined in each target plane, wherein the structural feature of the heart valve includes a fusion situation between any two adjacent leaflets of the heart valve; a second pushing reach point in each target plane is determined based on the structural feature of the heart valve, wherein the second pushing reach point is located at a boundary edge between the two adjacent leaflets; the target pattern is determined based on the first pushing reach point and the second pushing reach point, wherein the second pushing reach point is entirely located on the contour line of the target pattern.

Specifically, the method for determining the second pushing reach point varies depending on the fusion situation of the adjacent leaflets in the heart valve. In response to that a fusion fails to occur between the two adjacent leaflets, a junction point of the two adjacent leaflets is determined as the second pushing reach point corresponding to the two adjacent leaflets; In response to that the fusion occurs between the two adjacent leaflets, a second initial pushing point at a fusion area of the two adjacent leaflets is determined; and the second pushing reach point is determined based on the second initial pushing point.

In some embodiments of the present disclosure, in response to that the fusion occurs between the two adjacent leaflets, the method for determining the second pushing reach point based on the second initial pushing point is as follow: the fusion situation between the two adjacent leaflets is determined, wherein the fusion situation includes at least one of a fusion type and a length of a fused portion; a second pushing distance corresponding to the second initial pushing point is determined based on the fusion situation; and the second pushing reach point is determined based on the second pushing distance and the second initial pushing point, wherein the second pushing reach point corresponds to a position where the second initial pushing point moves the second pushing distance along the fused portion in a direction away from a center of the heart valve.

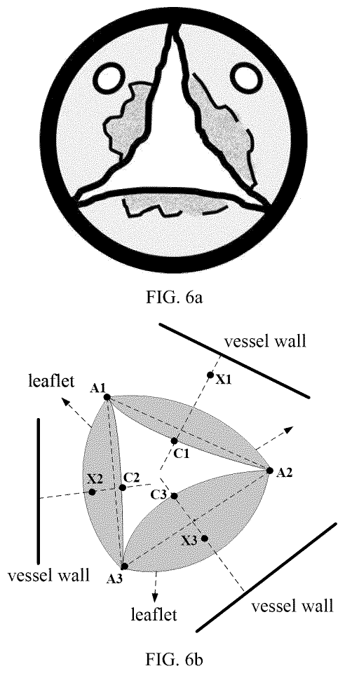

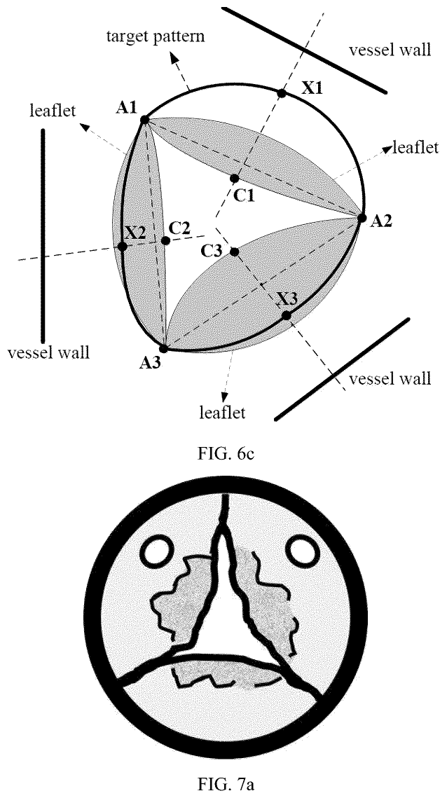

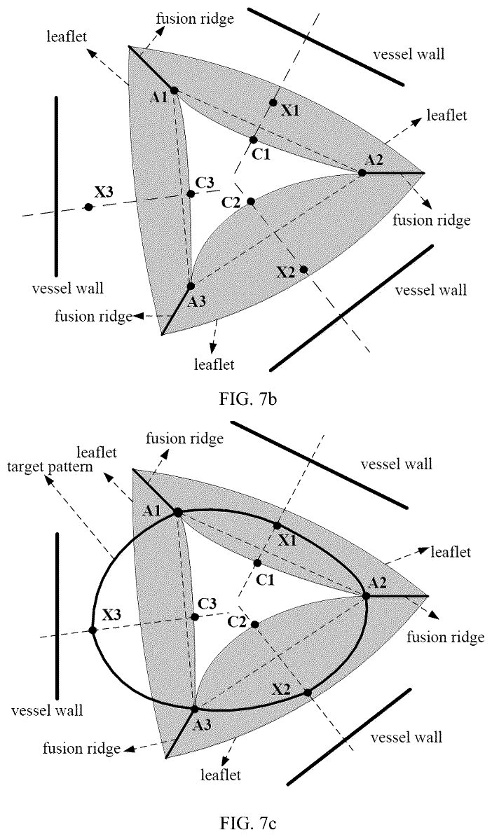

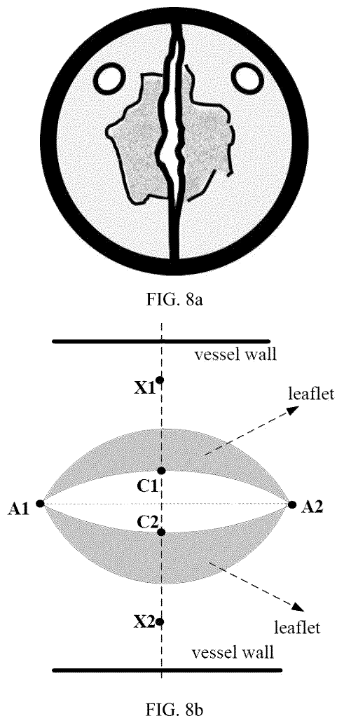

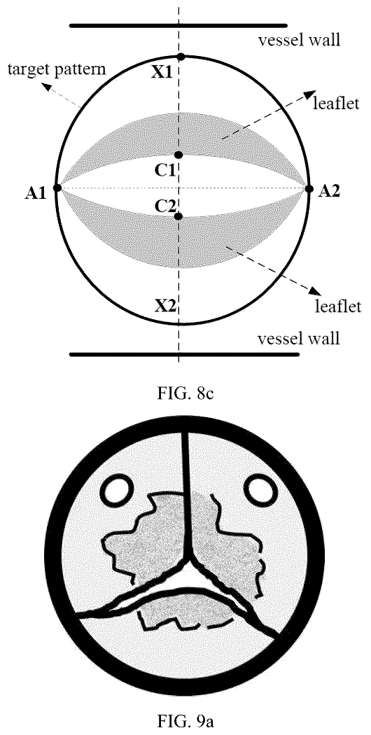

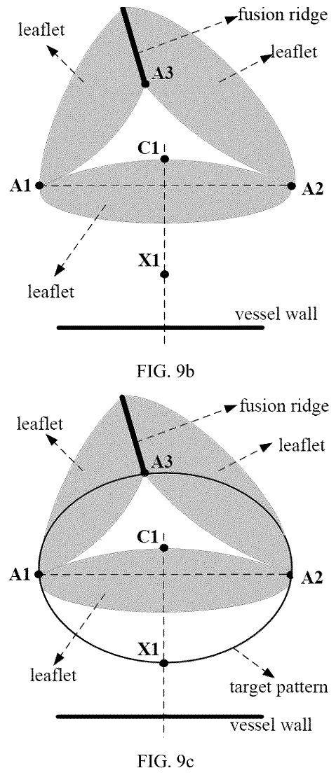

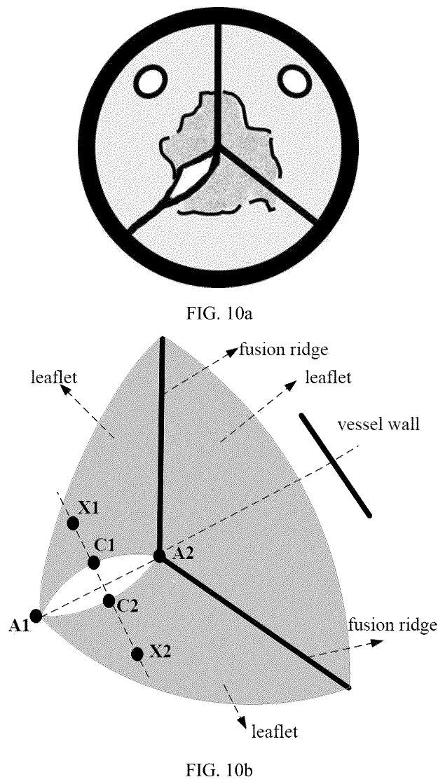

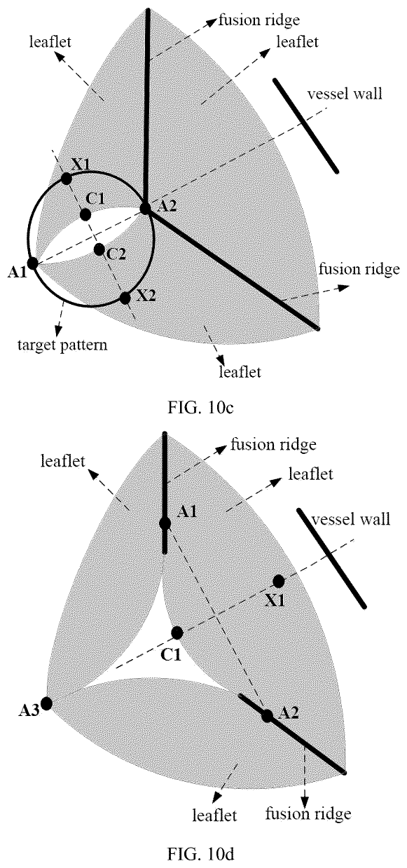

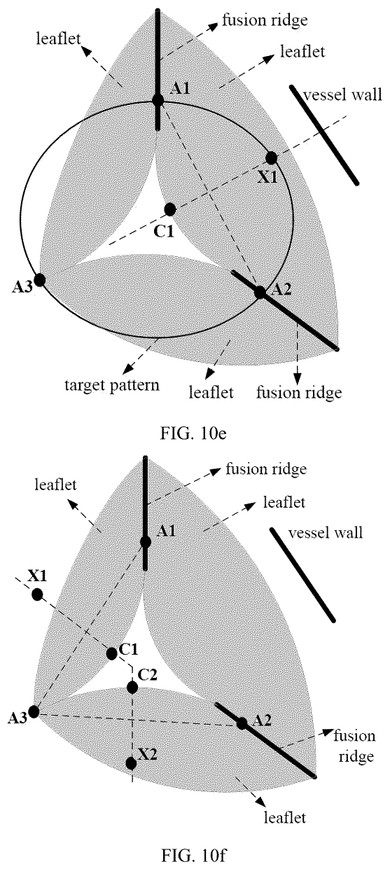

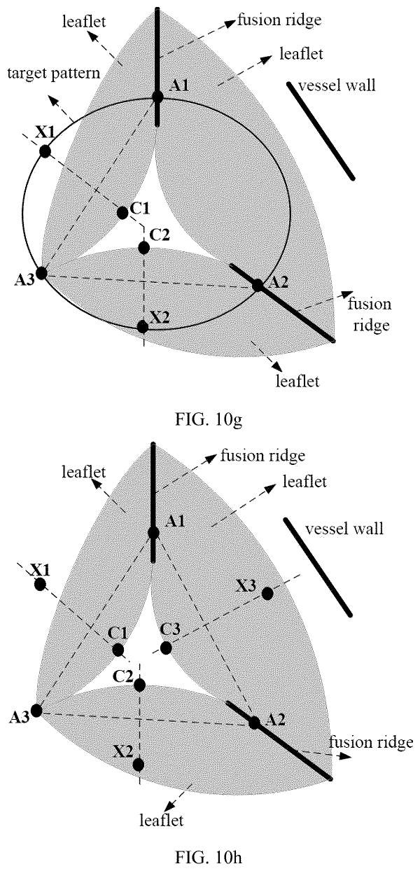

In some embodiments of the present disclosure, the heart valve can be classified into different types based on the number of leaflets and the degree of fusion between adjacent leaflets. Each type of heart valve corresponds to a specific method for determining a target pattern. The heart valve includes the first type of heart valve shown in FIG. 6a, the second type of heart valve shown in FIG. 7a, the third type of heart valve shown in FIG. 8a, the fourth type of heart valve shown in FIG. 9a, and the fifth type of heart valve shown in FIG. 10a. It should be noted that the above five types of representative valves are selected from clinical cases to further explain the present disclosure. However, this does not imply that the present disclosure is limited to these five types.

Specifically, for the first type of heart valve shown in FIG. 6a, the distinguishing feature is that the heart valve has three leaflets, and no fusion occurs between the leaflets. Alternatively, even if fusion occurs, the fusion length is less than a first predefined fusion length and can therefore be ignored. The first predefined fusion length can be set as required by the target subject.

The positions of the first initial pushing points, the first pushing reach points, and the second pushing reach points are shown in FIG. 6b. The number of first initial pushing points in the heart valve is three, corresponding to points C1, C2, and C3 in the figure. Similarly, the number of first pushing reach points corresponding to the first initial pushing points is also three, corresponding to points X1, X2, and X3 in the figure. Likewise, the number of second pushing reach points is three, corresponding to points A1, A2, and A3 in the figure.

As shown in FIG. 6b, during determining the first initial pushing points, the junction points of any two adjacent leaflets in the heart valve are first identified as base points. Then, three target line segments are formed by connecting the base points in pairs. Perpendicular bisectors are drawn for the three target line segments, and the intersection points of each perpendicular bisector with the inner side of the corresponding leaflet are identified as the first initial pushing points.

After determining the first initial pushing points, it is necessary to determine the pushing reach points corresponding to each first initial pushing point. Specifically, the first pushing distance for each first initial pushing point can be determined based on the thickness and calcification degree of the corresponding leaflet. The first pushing reach points are then determined by moving each first initial pushing point along the perpendicular bisector in the direction away from the center of the heart valve by the first pushing distance.

For the first type of heart valve, the base points mentioned above serve as the second pushing reach points.

Once the three first pushing reach points and the three second pushing reach points have been determined, the target pattern shown in FIG. 6c can be established based on these points, wherein all the first pushing reach points X1, X2, X3 and the second pushing reach points A1, A2, A3 are located on the contour line of the target pattern.

For the second type of heart valve shown in FIG. 7a, the distinguishing feature is that the heart valve has three leaflets, and fusion occurs between any two adjacent leaflets. This fusion involves adhesion and/or calcification of the leaflets, with the length of the adhesion and/or calcification, i.e., the fusion length, being no less than a first predefined fusion length and less than a second predefined fusion length.

The positions of the first initial pushing points, first pushing reach points, and second pushing reach points are shown in FIG. 7b. The number of first initial pushing points in the heart valve is three, corresponding to points C1, C2, and C3 in the figure. Similarly, the number of first pushing reach points is three, corresponding to points X1, X2, and X3. Likewise, the number of second pushing reach points is three, corresponding to points A1, A2, and A3.

As shown in FIG. 7b, during determining the first initial pushing points, the endpoints close to the leaflet inner side at the fusion area of any two adjacent leaflets are first identified as the second initial pushing points. The second pushing reach points are then determined by moving the second initial pushing points along the direction of fusion away from the center of the heart valve by a second pushing distance. The second pushing reach points are connected in pairs to form three target line segments, and perpendicular bisectors are drawn for these line segments. The intersection points of each perpendicular bisector with the inner side of the corresponding leaflet are identified as the first initial pushing points.

After determining the first initial pushing point, the first pushing distance corresponding to the first initial pushing point can be determined based on the leaflet thickness and calcification degree. The first initial pushing point then moves by the first pushing distance to reach the first pushing reach point.

Specifically, the method for determining the first pushing distance is the same as the method used for the first pushing distance in the first type of heart valve. For the second pushing distance, the fusion type and fusion length at the fusion area between the two adjacent leaflets are first determined. The pushing degree at the fusion area is then determined based on the fusion type, and the second pushing distance is calculated based on the pushing degree and the fusion length. The pushing degree can be represented by the fusion coefficient, and the second pushing distance equals the fusion length multiplied by the fusion coefficient.

The first pushing reach point is determined based on the first pushing distance, and the second pushing reach point is determined based on the second pushing distance. Specifically, the position of the first pushing reach point is the location where the first initial pushing point moves by the first pushing distance along the perpendicular bisector away from the center of the heart valve. The position of the second pushing reach point is the location where the second initial pushing point moves by the second pushing distance along the direction of fusion away from the center of the heart valve.

Once the three first pushing reach points and three second pushing reach points are determined, the target pattern shown in FIG. 7c can be established based on these points, wherein all the first pushing reach points X1, X2, X3 and second pushing reach points A1, A2, A3 are located on the contour line of the target pattern.

For the third type of heart valve shown in FIG. 8a, the distinguishing feature is that the heart valve has two leaflets, and no fusion occurs between them, or if fusion does occur, the fusion length is less than the first predefined fusion length, and therefore, it can be ignored. The first predefined fusion length can be set by the target subject.

For the third type of heart valve, as shown in FIG. 8b, the number of second pushing reach points (i.e., base points) is two. The junction points A1 and A2 at the roots of the two leaflets serve as the second pushing reach points.

As shown in FIG. 8b, during determining the first initial pushing points and the first pushing reach points, the two base points are connected to form the target line segment. Then, the intersection of the perpendicular bisector of the target line segment and the inner sides of the two leaflets serve as the first initial pushing points C1 and C2. The first pushing distance for each first initial pushing point is determined based on the thickness and calcification degree of the corresponding leaflet. Each first initial pushing point is then moved along the perpendicular bisector in the direction away from the center of the valve by the corresponding first pushing distance to reach the first pushing reach points X1 and X2.

Once the two first pushing reach points and two second pushing reach points are determined, the target pattern shown in FIG. 8c can be created, wherein all the first pushing reach points X1, X2 and second pushing reach points A1, A2 are located on the contour line of the target pattern.

For the fourth type of heart valve shown in FIG. 9a, the distinguishing feature is that the heart valve has three leaflets, but the fusion length between two of the leaflets is greater than the second predefined fusion length. Thus, this type of heart valve can be considered as a bicuspid valve, where one leaflet is normal, and the other leaflet is formed by fusion of two leaflets.

As shown in FIG. 9b, the number of first initial pushing points for the fourth type of heart valve is one, corresponding to point C1 in the figure. The number of first pushing reach points is one, corresponding to point X1 in the figure. The number of second pushing reach points is three, corresponding to points A1, A2, and A3 in the figure. Among these, A1 and A2 are the junction points of the two adjacent leaflets that have not undergone fusion, i.e., the base points.

During determining the first initial pushing point, first, the two base points are to form the target line segment. Then, the perpendicular bisector of the target line segment is drawn and the intersection of the bisector with the inner side of the target leaflet is determined as the first initial pushing point C1. The target leaflet is the leaflet that has not undergone fusion with other leaflets or whose fusion length is less than the first predefined fusion length.

Next, the first pushing distance is determined based on the thickness and calcification degree of the target leaflet. The first initial pushing point is then moved along the perpendicular bisector in the direction away from the valve center by the first pushing distance to reach the first pushing reach point.

To determine the second pushing reach point except for the two base points, the second initial pushing point corresponding to each second pushing reach point must first be determined. The endpoint close to the leaflet inner side at the fusion area of the two adjacent leaflets that have undergone fusion serves as the second initial pushing point, and the fusion length is greater than the second predefined fusion length. Once the second initial pushing point is identified, the second pushing distance is determined based on the fusion length and the pushing degree at the fusion area. The second initial pushing point is then moved along the fusion area in the direction away from the valve center by the second pushing distance to reach the second pushing reach point A3. The pushing degree is determined by the fusion type at the fusion area, which includes fully calcified fusion, partially calcified fusion, and non-calcified fusion.

After determining one first pushing reach point and three second pushing reach points, the target pattern shown in FIG. 9c can be determined, where the first pushing reach point X1 and the second pushing reach points A1, A2, A3 all lie on the contour line of the target pattern.

For the fifth type of heart valve shown in FIG. 10a, the distinguishing feature is that the heart valve has three leaflets, and one target leaflet has fused with the other two leaflets, with a fusion length greater than the first predefined fusion length. However, the other two leaflets have either not fused or their fusion length is less than the first predefined fusion length. In this case, the fifth type of heart valve is divided into four subtypes based on the fusion situation between the target leaflet and the other two leaflets.

Specifically, the first subtype of the fifth type of heart valve is characterized by complete fusion between the target leaflet and the other two leaflets, and the fused portion cannot be pushed apart. In this case, the initial pushing points and the pushing reach points for the first subtype are shown in FIG. 10b, including two first initial pushing points C1 and C2, two first pushing reach points X1 and X2, and two second pushing reach points A1 and A2. Among these, A1 is the junction point of the two leaflets that have not fused, and A2 is the junction point of the three leaflets.

During determining the first initial pushing points, the two second pushing reach points are connected to form a target line segment. Then, the perpendicular bisector of the target line segment is drawn, and the intersection of the perpendicular bisector with the inner side of the two non-target leaflets is determined to be the first initial pushing point. The first pushing distance is determined based on the thickness and fusion situation of the leaflets where the initial pushing points are located. Finally, the two first initial pushing points are moved along the perpendicular bisector towards the aortic sinus wall by the corresponding first pushing distance to determine the two first pushing reach points.

Once the two first pushing reach points and two second pushing reach points are determined, the target pattern shown in FIG. 10c can be created, wherein all the first pushing reach points X1, X2 and the second pushing reach points A1, A2 are located on the contour line of the target pattern.

The second subtype of the fifth type of heart valve is characterized by a fusion length between the target leaflet and the other two leaflets that is greater than the second predefined fusion length, but the fusion is not complete, and the fused portion can be partially pushed apart. In this case, the initial pushing points and pushing reach points for the second subtype are shown in FIG. 10d, including one first initial pushing point C1, one first pushing reach point X1, one second initial pushing point (i.e., second pushing reach point) A3, and two second pushing reach points A1 and A2.

Among these, the three second pushing reach points include A3, which is the junction point of the two adjacent leaflets that have not fused, while the second pushing reach points A1, A2 correspond to the second initial pushing point. The two second initial pushing points are located at the fusion area of the target leaflet and the other two leaflets, near the inner side of the leaflets. The method for determining the second pushing reach points corresponding to the second initial pushing points is the same as that used for the second type of heart valve, so it will not be repeated here.

During determining the first initial pushing point, the two second pushing reach points A1, A2 are first connected to form the target line segment. Then, the perpendicular bisector of the target line segment is drawn, and the intersection of the perpendicular bisector with the inner side of the target leaflet is determined as the first initial pushing point C1. After that, the method for determining the first pushing distance and the first pushing reach point is the same as the method used for the second type of heart valve and will not be repeated here.

After determining one first pushing reach point and three second pushing reach points, the target pattern shown in FIG. 10e can be determined, where the first pushing reach point X1 and the second pushing reach points A1, A2, A3 all lie on the contour line of the target pattern.

The third subtype of the fifth type of heart valve, compared to the second subtype, has the same fusion degree between the target leaflet and the other two leaflets. The only difference lies in the method of determining the first initial pushing point and the first pushing reach point. In these embodiments, both the first initial pushing points and the first pushing reach points are two in number.

Specifically, as shown in FIG. 10f, both the first initial pushing points and the first pushing reach points are two in number, and the second pushing reach points are three in number. Among the three second pushing reach points, A3 is the junction point of two adjacent leaflets that have not fused. The remaining two second pushing reach points A1, A2 correspond to the second initial pushing points. The two second initial pushing points are located at the fusion area of the target leaflet with the other two leaflets, near the inner side of the leaflets. The method for determining the second pushing reach points corresponding to the second initial pushing points is the same as the method used for the second type of heart valve, so it will not be repeated here.

To determine the first initial pushing points, the lines A1A3 and A2A3, two target line segments are first obtained as shown in FIG. 10f. Then, the perpendicular bisectors of these two line segments are drawn, and the intersections of the perpendicular bisectors with the inner sides of the remaining two leaflets (excluding the target leaflet) are determined as the first initial pushing points C1, C2. Afterward, the method for determining the first pushing distance and the first pushing reach points X1, X2 is the same as the method used for the second type of heart valve, and will not be repeated here.

Once the two first pushing reach points and three second pushing reach points are determined, the target pattern shown in FIG. 10g can be determined, where the first pushing reach points X1, X2 and the second pushing reach points A1, A2, A3 all lie on the contour line of the target pattern.

The fourth subtype of the fifth type of heart valve, compared to the second and third subtypes, has the same fusion degree between the target leaflet and the other two leaflets. The only difference lies in the method of determining the first initial pushing point and the first pushing reach point. In these embodiments, both the first initial pushing points and the first pushing reach points are three in number.

Specifically, as shown in FIG. 10h, both the first initial pushing points and the first pushing reach points are three in number, and the second pushing reach points are also three in number. Among the three second pushing reach points, A3 is the junction point of two adjacent leaflets that have not fused. The remaining two second pushing reach points A1, A2, correspond to the second initial pushing points. The two second initial pushing points are located at the fusion area of the target leaflet with the other two leaflets, near the inner side of the leaflets. The method for determining the second pushing reach points corresponding to the second initial pushing points is the same as the method used for the second type of heart valve, so it will not be repeated here.

During determining the first initial pushing points, the three second pushing reach points are connected pairwise to form three target line segments: A1A2, A1A3, and A2A3. The perpendicular bisectors of these line segments are then drawn, and the intersections of these bisectors with the inner sides of the leaflets serve as the first initial pushing points C1, C2, C3. Afterward, the method for determining the three first pushing reach points X1, X2, X3 is the same as the method used for the second type of heart valve, and will not be repeated here.

Once the three first pushing reach points X1, X2, X3 and the three second pushing reach points A1, A2, A3 are determined, the target pattern shown in FIG. 10i can be determined, where the first pushing reach points X1, X2, X3 and the second pushing reach points A1, A2, A3 all lie on the contour line of the target pattern.

In some embodiments of this disclosure, for the first to fifth types of heart valves, during determining the first pushing distance based on the thickness and calcification degree of the leaflets, the distance between the inner side of the leaflet where the first initial pushing point is located and the vessel wall can first be determined. Then, a distance coefficient is calculated based on the thickness and calcification degree of the leaflet. The first pushing distance is obtained by multiplying the distance between the leaflet inner side and the vessel wall by the distance coefficient.

Specifically, when the leaflet is calcified and the calcification extends to the aortic sinus wall, the leaflet is considered to be immovable, and the distance coefficient is 0. When the leaflet is thickened but does not fully occupy the aortic sinus, the distance coefficient can range from ⅓ to 7/10, with a preferred range of ½ to ⅔. Calcification of the leaflet can be classified into free edge calcification and solid filling calcification. Free edge calcification means that calcified material is attached to the edge of the leaflet or on the leaflet itself but does not extend to the aortic sinus wall. This type of calcification has minimal impact on the pushability of the leaflet, and the distance coefficient can range from ⅓ to 7/10, with a preferred range of ½ to ⅔. Solid filling calcification means that calcification is present on the leaflet and extends to the aortic sinus wall. In this case, the calcification fully fills the space between the leaflet and the aortic sinus, making the leaflet immovable. In such cases, the distance coefficient is 0.

For example, for the first type of heart valve, the distances between the three leaflets and the aortic sinus wall are measured as L1 equal to 13.7 mm, L2 equal to 12.6 mm, and L3 equal to 12.4 mm, and each leaflet has free edge calcification. Based on the above range of distance coefficients and combined with the actual anatomy of the aortic valve, the first pushing distance S1 corresponding to L1 is equal to 10 mm, with a distance coefficient of 73%; similarly, the first pushing distance S2 corresponding to L2 is equal to 8.3 mm, with a distance coefficient of 65.9%; and the first pushing distance S3 corresponding to L3 is equal to 7.1 mm, with a distance coefficient of 57.3%.

For the second type of heart valve, the distances between the two leaflets and the aortic sinus wall are measured as L1 equal to 11.9 mm, L2 equal to 12.4 mm, and L3 equal to 14.1 mm, and each leaflet has free edge calcification. Based on the above range of distance coefficients and combined with the actual anatomy of the aortic valve, the first pushing distance S1 corresponding to L1 is equal to 8 mm, with a distance coefficient of 67.2%; similarly, the first pushing distance S2 corresponding to L2 is equal to 6.3 mm, with a distance coefficient of 50.8%; and the first pushing distance S3 corresponding to L3 is equal to 7.7 mm, with a distance coefficient of 54.6%.

For the third type of heart valve, the distances between the leaflets and the aortic sinus wall are measured as L1 equal to 16.8 mm and L2 equal to 15.4 mm, and each leaflet has free edge calcification. Based on the above range of distance coefficients and combined with the actual anatomy of the aortic valve, the first pushing distance S1 corresponding to L1 is equal to 5.8 mm, with a distance coefficient of 34.5%; and the first pushing distance S2 corresponding to L2 is equal to 8.1 mm, with a distance coefficient of 52.6%.

For the fourth type of heart valve, the target leaflet that is not fused has free edge calcification, and the distance between the inner side of the non-fused target leaflet and the aortic sinus wall is L equal to 15.1 mm. Based on the above range of distance coefficients and combined with the actual anatomy of the aortic valve, the first pushing distance S corresponding to L is equal to 7.1 mm, with a distance coefficient of 47%. In another embodiment, the distance between the inner side of the target leaflet and the aortic sinus wall is L equal to 13 mm, and the first pushing distance S corresponding to L is equal to 5.9 mm, with a distance coefficient of 45.4%.

For the third subtype of the fifth type of heart valve, the other two leaflets, except for the target leaflet, have free edge calcification, and the distances between these leaflets and the aortic sinus wall are L1 equal to 14.9 mm and L2 equal to 8.2 mm. The target leaflet has fused with the other two leaflets. Based on the above range of distance coefficients and combined with the actual anatomy of the aortic valve, the first pushing distance S1 corresponding to L1 is equal to 6.3 mm, with a distance coefficient of 42.3%; and the first pushing distance S2 corresponding to L2 is equal to 3.7 mm, with a distance coefficient of 45.1%.

When determining the second pushing distance, different fusion types correspond to different degrees of pushing degree, which can be represented by the fusion coefficient. Specifically, when the fusion type is fully calcified fusion, the pushing degree, or fusion coefficient, is 0; when the fusion type is partially calcified fusion, the fusion coefficient can range from ⅓ to ⅔, preferably between ⅖ and ⅗. In this case, the fusion length at the fusion area is the actual fusion length minus the length of the calcified part. When multiple calcification sites are present at the fusion area, the length of the calcified part is the sum of the lengths of the individual calcification sites. When the fusion type is non-calcified fusion, the fusion coefficient can range from ⅓ to ⅔, preferably between ⅖ and ⅗.

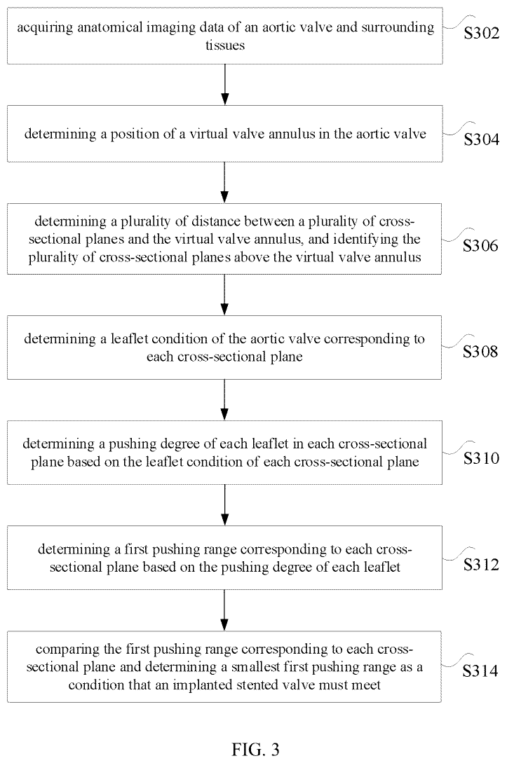

FIG. 3 is a schematic flowchart of a process for determining the heart valve according to one embodiment of the present disclosure. As shown in FIG. 3, the process includes the following steps:

S302, acquiring anatomical imaging data of an aortic valve and surrounding tissues; and

S304, determining a position of a virtual valve annulus in the aortic valve;

In this embodiment, the virtual valve annulus corresponds to a reference plane, which is a plane determined by a lowest point where a aortic valve leaflet is attached to a vessel wall.

S306, determining a plurality of distance between a plurality of cross-sectional planes and the virtual valve annulus, and identifying the plurality of cross-sectional planes above the virtual valve annulus;

S308, determining a leaflet condition of the aortic valve corresponding to each cross-sectional plane;

S310, determining a pushing degree of each leaflet in each cross-sectional plane based on the leaflet condition of each cross-sectional plane;

S312, determining a first pushing range corresponding to each cross-sectional plane based on the pushing degree of each leaflet; and

S314, comparing the first pushing range corresponding to each cross-sectional plane and determining a smallest first pushing range as a condition that an implanted stented valve must meet.

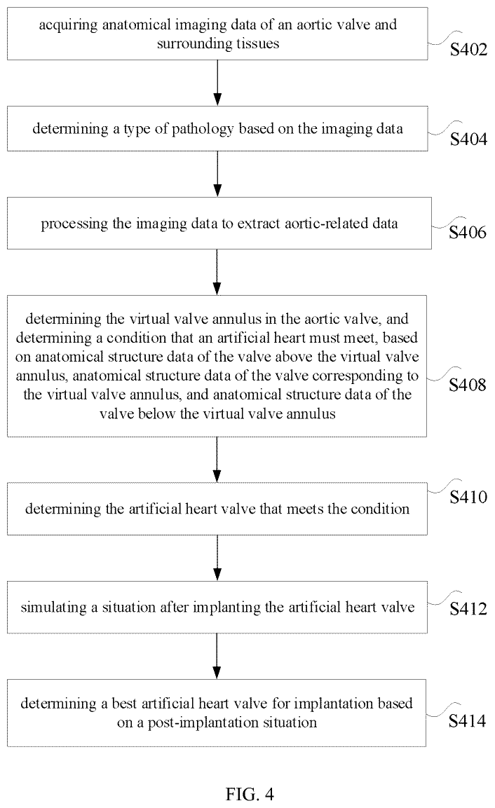

FIG. 4 is a schematic flowchart of the process for determining another type of heart valve according to one embodiment of the present disclosure. The process includes the following steps:

S402, acquiring anatomical imaging data of an aortic valve and surrounding tissues;

In some embodiments of the present disclosure, the above imaging data can be acquired through ultrasound, computed tomography (CT), nuclear magnetic resonance (MRI), etc.

S404, determining a type of pathology based on the imaging data;

In some embodiments, the pathology types include aortic stenosis and regurgitation.

S406, processing the imaging data to extract aortic-related data;

In some embodiments, processing the imaging data includes remodelling the structural information of the aorta and surrounding tissues in the imaging data.

S408, determining the virtual valve annulus in the aortic valve, and determining a condition that an artificial heart must meet, based on anatomical structure data of the valve above the virtual valve annulus, anatomical structure data of the valve corresponding to the virtual valve annulus, and anatomical structure data of the valve below the virtual valve annulus;

S410, determining the artificial heart valve that meets the condition; S412, simulating a situation after implanting the artificial heart valve; and

S414, determining a best artificial heart valve for implantation based on a post-implantation situation.

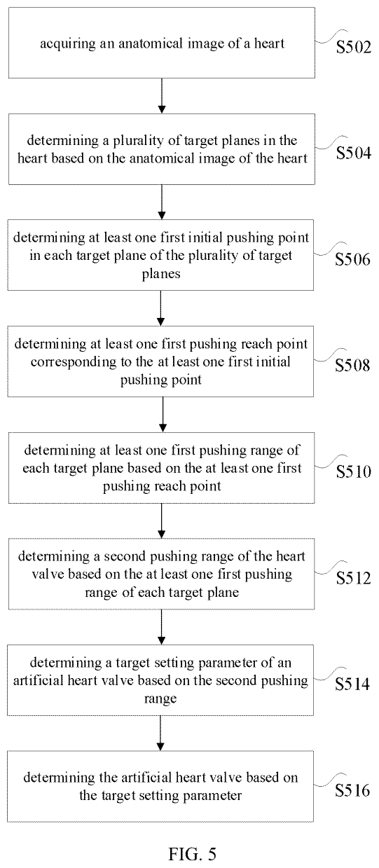

FIG. 5 is a schematic flowchart of another method for determining the heart valve based on multiple planes according to one embodiment of the present disclosure. The method includes the following steps:

S502, acquiring an anatomical image of a heart;

S504, determining a plurality of target planes in the heart based on the anatomical image of the heart;

S506, determining at least one first initial pushing point in each target plane of the plurality of target planes, wherein each first initial pushing point is a point located on an inner contour line of a heart valve leaflet;

S508, determining at least one first pushing reach point corresponding to the at least one first initial pushing point, wherein each first pushing reach point corresponds to a position where a corresponding first initial pushing point reaches after moving a first pushing distance towards a vessel wall, and the vessel wall is a wall of an aortic sinus in the heart;

S510, determining at least one first pushing range of each target plane based on the at least one first pushing reach point;

S512, determining a second pushing range of the heart valve based on the at least one first pushing range of each target plane;

S514, determining a target setting parameter of an artificial heart valve based on the second pushing range, wherein the target setting parameter includes dimensional information of the artificial heart valve; and

S516, determining the artificial heart valve based on the target setting parameter.

In the technical solution provided in step S514, the target planes are all located above the aortic valve annulus. To ensure that the final determined artificial heart valve meets the actual requirements, as an optional embodiment, during determining the target setting parameters for the artificial heart valve, a first structural feature of the aortic valve annulus is determined and a second structural feature of a heart valve below the aortic valve annulus is determined; and the target setting parameter is determined based on the first structural feature, the second structural feature and the second pushing range.

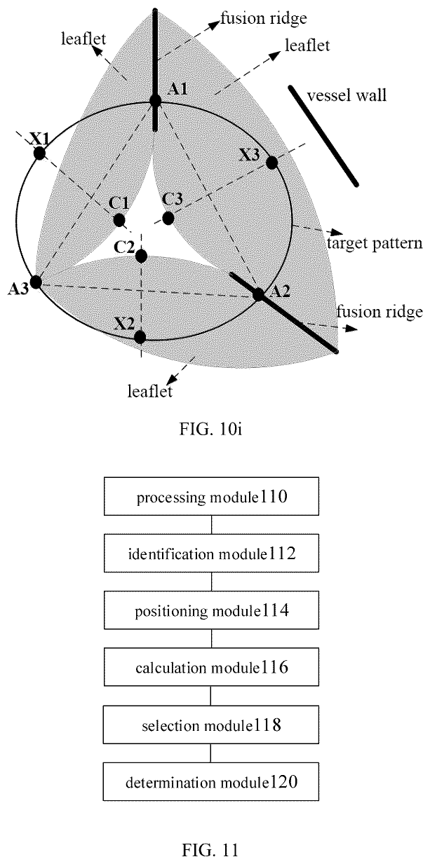

According to the embodiments of the present disclosure, an apparatus for determining a heart valve based on multiple planes is provided. FIG. 11 is a schematic structural view of an apparatus for determining a heart valve based on multiple planes according to a embodiment of the present disclosure. As shown in FIG. 11, the apparatus includes: a processing module 110, configured to determine a plurality of target planes in a heart based on an anatomical image of the heart; an identification module 112, configured to determine at least one first initial pushing point in each target plane of the plurality of target planes, wherein each first initial pushing point is a point located on an inner contour line of a heart valve leaflet; a positioning module 114, configured to determine at least one first pushing reach point corresponding to the at least one first initial pushing point, wherein each first pushing reach point corresponds to a position where a corresponding first initial pushing point reaches after moving a first pushing distance towards a vessel wall, and the vessel wall is a wall of an aortic sinus in the heart; a calculation module 116, configured to determine at least one first pushing range of each target plane based on the at least one first pushing reach point; a selection module 118, configured to determine a second pushing range of the heart valve based on the at least one first pushing range of each target plane; and a determination module 120, configured to determine an artificial heart valve that matches the heart valve based on the second pushing range.

It should be noted that the apparatus shown in FIG. 11 can be used to perform the method for determining a heart valve based on multiple planes shown in FIG. 1. Therefore, the relevant explanations of the method shown in FIG. 1 also apply to the embodiments of the present disclosure and will not be repeated here.

According to the embodiments of the present disclosure, a non-volatile storage medium is provided. The non-volatile storage medium includes a stored program, wherein when the program is executed, a device including the non-volatile storage medium is controlled to perform the method for determining the heart valve based on multiple planes including steps of: determining a plurality of target planes in a heart based on an anatomical image of the heart; determining at least one first initial pushing point in each target plane of the plurality of target planes, wherein each first initial pushing point is a point located on an inner contour line of a heart valve leaflet; determining at least one first pushing reach point corresponding to the at least one first initial pushing point, wherein each first pushing reach point corresponds to a position where a corresponding first initial pushing point reaches after moving a first pushing distance towards a vessel wall, and the vessel wall is a wall of an aortic sinus in the heart; determining at least one first pushing range of each target plane based on the at least one first pushing reach point; determining a second pushing range of the heart valve based on the at least one first pushing range of each target plane; and determining an artificial heart valve that matches the heart valve based on the second pushing range.

As an optional embodiment, when the above-mentioned program is executed, the device where the storage medium is located may also be controlled to execute the method for determining the heart valve based on multiple planes including steps of: acquiring an anatomical image of a heart; determining a plurality of target planes in the heart based on the anatomical image of the heart; determining at least one first initial pushing point in each target plane of the plurality of target planes, wherein each first initial pushing point is a point located on an inner contour line of a heart valve leaflet; determining at least one first pushing reach point corresponding to the at least one first initial pushing point, wherein each first pushing reach point corresponds to a position where a corresponding first initial pushing point reaches after moving a first pushing distance towards a vessel wall, and the vessel wall is a wall of an aortic sinus in the heart; determining at least one first pushing range of each target plane based on the first pushing reach point; determining a second pushing range of the heart valve based on the at least one first pushing range of each target plane; determining a target setting parameter of an artificial heart valve based on the second pushing range, wherein the target setting parameter includes dimensional information of the artificial heart valve; and determining the artificial heart valve based on the target setting parameter.

According to the embodiments of the present disclosure, an electronic device is provided, which includes a processor. The processor is configured to run a program, and when the program is executed, the method for determining the heart valve based on multiple planes is implemented. The method includes steps of: determining a plurality of target planes in a heart based on an anatomical image of the heart; determining at least one first initial pushing point in each target plane of the plurality of target planes, wherein each first initial pushing point is a point located on an inner contour line of a heart valve leaflet; determining at least one first pushing reach point corresponding to the at least one first initial pushing point, wherein each first pushing reach point corresponds to a position where a corresponding first initial pushing point reaches after moving a first pushing distance towards a vessel wall, and the vessel wall is a wall of an aortic sinus in the heart; determining at least one first pushing range of each target plane based on the at least one first pushing reach point; determining a second pushing range of the heart valve based on the at least one first pushing range of each target plane; and determining an artificial heart valve that matches the heart valve based on the second pushing range.

As an optional embodiment, when the above-mentioned program is executed, the device where the storage medium is located may also be controlled to perform the method for determining the heart valve based on multiple planes including steps of: acquiring an anatomical image of a heart; determining a plurality of target planes in the heart based on the anatomical image of the heart; determining at least one first initial pushing point in each target plane of the plurality of target planes, wherein each first initial pushing point is a point located on an inner contour line of a heart valve leaflet; determining at least one first pushing reach point corresponding to the at least one first initial pushing point, wherein each first pushing reach point corresponds to a position where a corresponding first initial pushing point reaches after moving a first pushing distance towards a vessel wall, and the vessel wall is a wall of an aortic sinus in the heart; determining at least one first pushing range of each target plane based on the first pushing reach point; determining a second pushing range of the heart valve based on the at least one first pushing range of each target plane; determining a target setting parameter of an artificial heart valve based on the second pushing range, wherein the target setting parameter includes dimensional information of the artificial heart valve; and determining the artificial heart valve based on the target setting parameter.

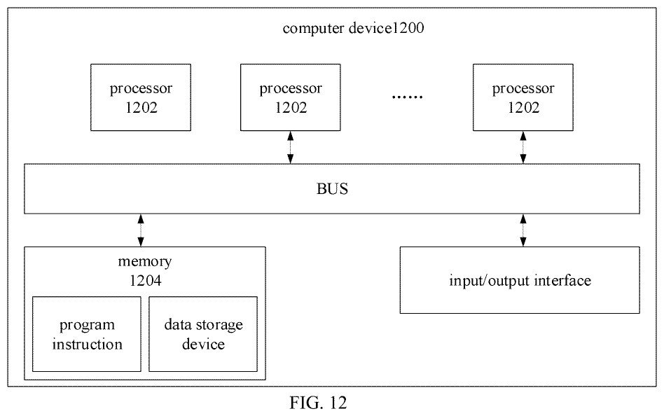

According to an embodiment of the present disclosure, a computer terminal is also provided. FIG. 12 is a schematic structural view of a computer device 1200 according to one embodiment of the present disclosure.

In some embodiments, a computer-readable storage medium including instructions is also provided, such as a memory 1204 containing instructions. These instructions can be executed by the processor 1202 of the device 1200 to perform the method for determining the heart valve based on multiple planes including steps of: determining a plurality of target planes in a heart based on an anatomical image of the heart; determining at least one first initial pushing point in each target plane of the plurality of target planes, wherein each first initial pushing point is a point located on an inner contour line of a heart valve leaflet; determining at least one first pushing reach point corresponding to the at least one first initial pushing point, wherein each first pushing reach point corresponds to a position where a corresponding first initial pushing point reaches after moving a first pushing distance towards a vessel wall, and the vessel wall is a wall of an aortic sinus in the heart; determining at least one first pushing range of each target plane based on the at least one first pushing reach point; determining a second pushing range of the heart valve based on the at least one first pushing range of each target plane; and determining an artificial heart valve that matches the heart valve based on the second pushing range. Optionally, the storage medium can be a non-transitory computer-readable storage medium. For example, the non-transitory computer-readable storage medium can be read-only memory, (ROM), random access memory (RAM), compact disc read-only memory (CD-ROM), magnetic tape, floppy disk, optical data storage devices, and so on.

As an optional embodiment, these instructions can be executed by the processor 1202 of the device 1200 to perform the method for determining the heart valve based on multiple planes including steps of: acquiring an anatomical image of a heart; determining a plurality of target planes in the heart based on the anatomical image of the heart; determining at least one first initial pushing point in each target plane of the plurality of target planes, wherein each first initial pushing point is a point located on an inner contour line of a heart valve leaflet; determining at least one first pushing reach point corresponding to the at least one first initial pushing point, wherein each first pushing reach point corresponds to a position where a corresponding first initial pushing point reaches after moving a first pushing distance towards a vessel wall, and the vessel wall is a wall of an aortic sinus in the heart; determining at least one first pushing range of each target plane based on the first pushing reach point; determining a second pushing range of the heart valve based on the at least one first pushing range of each target plane; determining a target setting parameter of an artificial heart valve based on the second pushing range, wherein the target setting parameter includes dimensional information of the artificial heart valve; and determining the artificial heart valve based on the target setting parameter.

In the above embodiments of the present disclosure, the descriptions of each embodiment are focused on different aspects. Any parts not elaborated in one embodiment can be referenced in the related descriptions of other embodiments.

In the several embodiments provided in this disclosure, it should be understood that the disclosed technical content can be implemented in other ways. The device embodiments described above are merely illustrative. For example, the divisions of the units may represent logical functional divisions, and other divisions may be used in practical implementations. For instance, multiple units or components may be combined or integrated into another system, or some features may be omitted or not executed. Additionally, the couplings or direct couplings or communication connections shown or discussed between units or components may be through interfaces, or the indirect coupling or communication connection between units or modules can be in electrical or other forms.

The units described as separate components may or may not be physically separated. The components shown as units may or may not be physical units, meaning they could be located in one place or distributed across multiple units. Depending on actual needs, some or all of the units can be selected to achieve the objectives of the present embodiment.

Furthermore, in each embodiment of the present disclosure, the functional units may be integrated into one processing unit, or the units may exist separately, or two or more units may be integrated into one unit. The integrated units can be implemented in hardware form or in the form of software functional units.

If the integrated units are implemented as software functional units and sold or used as independent products, they can be stored in a computer-readable storage medium. Based on this understanding, the technical solution of the present disclosure, or part of it, or the entire solution, may be embodied in the form of a software product. This computer software product is stored in a storage medium and includes several instructions that cause a computer device (such as a personal computer, server, or network device) to perform all or part of the steps described in the embodiments of the present disclosure. The aforementioned storage mediums include USB drives, read-only memory (ROM), random access memory (RAM), external hard drives, floppy disks, optical disks, and other media that can store program codes.

The above is only a preferred embodiment of the present disclosure. It should be noted that for those skilled in the art, various improvements and modifications can be made without departing from the principles of the present disclosure. These improvements and modifications should also be considered within the scope of protection of the present disclosure.

What is claimed is:

1. A method for determining a heart valve based on multiple planes, comprising steps of:
determining a plurality of target planes in a heart based on an anatomical image of the heart;
determining at least one first initial pushing point in each target plane of the plurality of target planes, wherein each first initial pushing point is a point located on an inner contour line of a heart valve leaflet;
determining at least one first pushing reach point corresponding to the at least one first initial pushing point, wherein each first pushing reach point corresponds to a position where a corresponding first initial pushing point reaches after moving a first pushing distance towards a vessel wall, and the vessel wall is a wall of an aortic sinus in the heart;
determining at least one first pushing range of each target plane based on the at least one first pushing reach point;
determining a second pushing range of the heart valve based on the at least one first pushing range of each target plane; and
determining an artificial heart valve that matches the heart valve based on the second pushing range.

2. The method for determining the heart valve based on multiple planes according to claim 1, wherein the step of determining a plurality of the target planes in a heart based on the anatomical image of the heart comprises steps of:
determining a position of a reference plane in the heart based on the anatomical image; and
selecting a plurality of cross-sectional planes above the reference plane, wherein the plurality of cross-sectional planes is defined as the plurality of target planes.

3. The method for determining the heart valve based on multiple planes according to claim 2, wherein the reference plane is a plane determined by a lowest point where the heart valve leaflet attaches to the wall of the aortic sinus.

4. The method for determining the heart valve based on multiple planes according to claim 1, wherein the step of determining at least one first pushing range of each target plane based on the at least one first pushing reach point comprises steps of:
determining a target pattern based on the at least one first pushing reach point, wherein each first pushing reach point is located on a contour line of the target pattern; and
determining size information of the target pattern and determining the at least one first pushing range based on the size information.

5. The method for determining the heart valve based on multiple planes according to claim 4, wherein the size information of the target pattern comprises at least one of a perimeter, an area, a radius, a diameter, and an average diameter of the target pattern.

6. The method for determining the heart valve based on multiple planes according to claim 4, wherein the step of determining a target pattern based on the at least one first pushing reach points comprises steps of:
determining a structural feature of the heart valve in each target plane, wherein the structural feature of the heart valve comprises a fusion situation between any two adjacent leaflets of the heart valve;
determining at least one second pushing reach point in each target plane based on the structural feature of the heart valve, wherein each second pushing reach point is located at a boundary edge between the two adjacent leaflets; and
determining the target pattern based on the at least one first pushing reach point and the at least one second pushing reach point, wherein each second pushing reach point is located on the contour line of the target pattern.

7. The method for determining the heart valve based on multiple planes according to claim 6, wherein the step of determining at least one second pushing reach point in each target plane based on the structural feature of the heart valve comprises steps of:
determining a junction point of the two adjacent leaflets as a second pushing reach point corresponding to the two adjacent leaflets in case of no fusion between the two adjacent leaflets;
determining a second initial pushing point at a fusion area of the two adjacent leaflets in case of fusion occurring between the two adjacent leaflets; and
determining a second pushing reach point based on the second initial pushing point.

8. The method for determining the heart valve based on multiple planes according to claim 7, wherein the step of determining a second pushing reach point based on the second initial pushing point comprises steps of:
determining the fusion situation between the two adjacent leaflets, wherein the fusion situation comprises at least one of a fusion type and a length of a fused portion;
determining a second pushing distance corresponding to the second initial pushing point based on the fusion situation; and
determining the second pushing reach point based on the second pushing distance and the second initial pushing point, wherein the second pushing reach point corresponds to a position where the second initial pushing point reaches after moving the second pushing distance along the fused portion in a direction away from a center of the heart valve.

9. The method for determining the heart valve based on multiple planes according to claim 8, wherein the step of determining the fusion situation between the two adjacent leaflets comprises:
determining the fusion type and the length of the fused portion between two adjacent leaflets; and
wherein the step of determining a second pushing distance corresponding to the second initial pushing point based on the fusion situation comprises steps of:
determining a pushing degree at the fusion area based on the fusion type; and
determining the second pushing distance based on the pushing degree and the length of the fused portion.

10. The method for determining the heart valve based on multiple planes according to claim 6, wherein the fusion situation comprises at least one of a fusion type and a length of a fused portion, and the fusion type comprises fully calcified fusion, partially calcified fusion, and non-calcified fusion.

11. The method for determining the heart valve based on multiple planes according to claim 1, wherein the step of determining a second pushing range of the heart valve based on the at least one first pushing range of each target plane comprises:
determining a smallest first pushing range of the at least one first pushing range as the second pushing range.

12. The method for determining the heart valve based on multiple planes according to claim 1, wherein the step of determining at least one first pushing reach point corresponding to the at least one first initial pushing point comprises steps of:
determining a thickness and a calcification degree of the leaflet corresponding to each first initial pushing point;
determining each first pushing distance based on the thickness and the calcification degree of the leaflet; and
determining a position of each first pushing reach point based on a position of each first initial pushing point and each first pushing distance.

13. The method for determining the heart valve based on multiple planes according to claim 12, the step of determining each first pushing distance based on the thickness and the calcification degree of the leaflet comprises steps of:
determining a distance between an inner side of the leaflet where each first initial pushing point is located and the vessel wall; and
determining a distance coefficient based on the thickness and calcification degree of the leaflet, and calculating each first pushing distance by multiplying the distance between the inner side of the leaflet and the vessel wall by the distance coefficient.

14. The method for determining the heart valve based on multiple planes according to claim 1, wherein each target plane is perpendicular to a central axis of an aorta of the heart.

15. The method for determining the heart valve based on multiple planes according to claim 1, wherein the anatomical image comprises structures of an aortic valve and surrounding tissues, and each target plane comprises contour information of the heart valve and the surrounding tissues.

16. A method for determining the heart valve based on multiple planes, comprising steps of:
acquiring an anatomical image of a heart;
determining a plurality of target planes in the heart based on the anatomical image of the heart;
determining at least one first initial pushing point in each target plane of the plurality of target planes, wherein each first initial pushing point is a point located on an inner contour line of a heart valve leaflet;
determining at least one first pushing reach point corresponding to the at least one first initial pushing point, wherein each first pushing reach point corresponds to a position where a corresponding first initial pushing point reaches after moving a first pushing distance towards a vessel wall, and the vessel wall is a wall of an aortic sinus in the heart;
determining at least one first pushing range of each target plane based on the at least one first pushing reach point;
determining a second pushing range of the heart valve based on the at least one first pushing range of each target plane;
determining a target setting parameter of an artificial heart valve based on the second pushing range, wherein the target setting parameter comprises dimensional information of the artificial heart valve; and
determining the artificial heart valve based on the target setting parameter.

17. The method for determining the heart valve based on multiple planes according to claim 16, wherein the target plane is a plane located above an aortic valve annulus, and the step of determining a target setting parameter of an artificial heart valve based on the second pushing range comprises:
determining a first structural feature of the aortic valve annulus and a second structural feature of a heart valve below the aortic valve annulus; and
determining the target setting parameter based on the first structural feature, the second structural feature and the second pushing range.

18. An apparatus for determining a heart valve based on multiple planes, comprising:
a processing module, configured to determine a plurality of target planes in a heart based on an anatomical image of the heart;
an identification module, configured to determine at least one first initial pushing point in each target plane of the plurality of target planes, wherein each first initial pushing point is a point located on an inner contour line of a heart valve leaflet;
a positioning module, configured to determine at least one first pushing reach point corresponding to the at least one first initial pushing point, wherein each first pushing reach point corresponds to a position where a corresponding first initial pushing point reaches after moving a first pushing distance towards a vessel wall, and the vessel wall is a wall of an aortic sinus in the heart;
a calculation module, configured to determine at least one first pushing range of each target plane based on the at least one first pushing reach point;
a selection module, configured to determine a second pushing range of the heart valve based on the at least one first pushing range of each target plane; and
a determination module, configured to determine an artificial heart valve that matches the heart valve based on the second pushing range.

19. A non-volatile storage medium, comprising a stored program, wherein when the program is executed, a device comprising the non-volatile storage medium is controlled to perform the method for determining the heart valve based on multiple planes according to claim 1.

20. An electronic device, comprising a processor, wherein the processor is configured to execute a program, and when the program is executed, the method for determining the heart valve based on multiple planes according to claim 1 is implemented.
